(19) United States
(12) Patent Application Publication
(10) Pub. No.: US 2009/0062406 A1
(43) Pub. Date: Mar. 5, 2009

(54) Aqueous concentrates of isethionate, taurate and betaine

(75) Inventor: Matthias Loeffler, Idstein (DE)

Correspondence Address: CLARIANT CORPORATION;INTELLECTUAL PROPERTY DEPARTMENT, 4000 MONROE ROAD, CHARLOTTE, NC, 28205, US

(73) Assignee: Clariant International Ltd.

(21) Appl. No.: 12/231,209

(22) Filed: Aug. 29, 2008

(30) Foreign Application Priority Data
Aug. 30, 2007 (DE) 102007040909.7

(51) Int. Cl. A61K 8/46; A61K 47/20

(52) U.S. Cl. 514/785; 510/119; 510/130

(57) ABSTRACT

The invention provides aqueous concentrates comprising, based on the total composition,
A) 0.1 to 8.0% by weight, preferably 3.0 to 7.0% by weight, of one or more isethionates of formula (1)

RCOOCH₂CH₂SO₃M (1)

in which
R is a linear or branched alkyl group or alkenyl group and
M is a counterion,
B) 0.1 to 8.0% by weight of one or more taurates of formula (2)

R¹CON(CH₃)CH₂CH₂SO₃X (2)

in which R¹ and X have the same meaning as R and M and
C) 0.1 to 40.0% by weight of one or more alkylbetaines of formula (3)

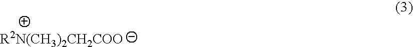

in which R² has the same meaning as R, with the proviso that the concentration of the sum of all surfactants with the formula 1, 2 and 3 is more than 20% by weight.

These concentrates are flowable at room temperature and are transparent and also colorless.

The invention relates to highly concentrated, transparent and colorless, aqueous concentrates comprising isethionates, taurates and alkylbetaine that are flowable at room temperature.

Isethionates and taurates are known as mild surfactants with widespread use in cosmetic compositions.

Sodium methyl cocoyl taurate is an anionic surfactant which, on account of good skin compatibility and good foam properties, is used in particular as cosurfactant in foaming face cleansing compositions. However, taurates are only slightly soluble in water. The solubility of sodium methyl cocoyl taurate in water is 10 g/l (20° C.). In contrast to the very much better water-soluble surfactants, such as, for example, sodium laureth sulfate or cocamidopropylbetaine (which are available as transparent, thin-liquid 30% strength aqueous solution e.g. under the Clariant trade names Genapol LRO liquid or Genagen CAB), taurates in a comparable concentration in water are neither transparent nor liquid. Thus, the typical commercial form of sodium methyl cocoyl taurate is a 30% strength paste which can only be removed from the packs with difficulty and categorically requires hot processing. Example: Hostapon CT paste (Clariant Produkte (Deutschland) GmbH).

Sodium cocoyl isethionate is an anionic surfactant which, on account of its good foam properties and in particular because of its very low solubility in water, has been used for many years as a raw material in the manufacture of bar soaps (combination bar or syndet). The solubility of sodium cocoyl isethionate in water is only 0.01% (20° C.). Isethionates are therefore typically available in the marketplace as solid products. Example: Hostapon SCI 85 powder. For processing in cosmetic compositions, the solid raw material has to be dissolved hot using surfactants or solvents with high energy and time expenditure. The obligatorily hot processing also hinders the incorporation of temperature-sensitive additives, e.g. vitamins, dyes and the like. Processing the solid isethionate often results in troublesome dusting of the raw material, which further increases the expenditure upon use, for example suitable protective clothing is required or it is necessary to work in special sealed plants.

Surfactant concentrates comprising sparingly soluble surfactants, e.g. sodium cocoyl isethionate, in the presence of amphoteric surfactants are known. In these concentrates, ethylene glycol mono- and/or distearates, which are essentially insoluble in water, are stably dispersed with the help of suitable surfactants such that an opaque dispersion is present at room temperature. U.S. Pat. No. 5,529,721, U.S. Pat. No. 4,777,038 and U.S. Pat. No. 5,560,873 describe such concentrates, which are also available under the trade name Mirasheen® (Rhone-Poulenc) or Euperlan (HENKEL).

U.S. Pat. No. 5,415,810 describes mixtures comprising amphoteric surfactants in the presence of sodium cocoyl isethionate. However, there is no indication that these mixtures are clear and stable. Rather, these mixtures are added directly after preparation to a cosmetic product in which the isethionate is dissolved and stabilized by additional known additives and in which the isethionate and the other surfactants of the mixture are diluted with water.

In addition, U.S. Pat. No. 5,415,810 does not mention the combination of sodium cocoyl isethionate with other sparingly soluble surfactants in the concentrate. In contrast to this, it is also expressly advised that sparingly soluble components such as fatty acids should not be present in the concentrates.

U.S. Pat. No. 4,243,549 describes concentrates comprising anionic and amphoteric surfactants although these comprise essentially more than 60% by weight of surfactant and thus form a lamellar or “G” phase. In addition, U.S. Pat. No. 4,243,549 teaches the use of readily water-soluble surfactants, such as, for example, sodium laureth sulfate.

Sparingly soluble surfactants, and in particular mixtures of sparingly soluble surfactants such as sodium cocoyl isethionate and sodium methyl cocoyl taurate are not taught.

Other literature (e.g. U.S. Pat. No. 5,372,751, U.S. Pat. No. 5,518,647, EP-692 240) describes the use of sodium cocoyl isethionate in combination with amphoteric surfactants and additional anionic surfactant. The surfactant mixtures described therein are only liquid at low surfactant concentrations; at high surfactant concentrations, these surfactant mixtures are solid or pasty, and not liquid and clear. The combination of two sparingly soluble anionic surfactants (sodium cocoyl isethionate and sodium methyl cocoyl taurate) in cocobetaine is not disclosed.

Surprisingly, it has now been found that aqueous mixtures of isethionates, taurates and betaines, even in the case of a high surfactant content, are clear, homogeneous, flowable and pumpable concentrates which additionally also have very low color numbers. Furthermore, it has been found that the use of cocobetaine as amphoteric surfactant permits the solubilization of a mixture of both taurates and also isethionates in water in a considerably higher concentration than would be possible in the absence of the amphoteric surfactant. Furthermore, it has been found that the presence of further sparingly soluble surfactants, such as, for example, fatty acids (which are present typically up to 35% as secondary constituent in isethionates) does not alter the clear, homogeneous, flowable and pumpable state of the concentrates.

Surprisingly, it has also been found that the surfactant concentrates according to the invention at the same use concentration maintain better foam behavior than the individual components. The presence of a synergistic mixture in the surfactant concentrates according to the invention was demonstrated by foam measurements (Ross Miles) and handwashing tests using an expert panel.

The invention provides transparent, flowable, colorless aqueous concentrates comprising, based on the total concentrate,
A) 0.1 to 8.0% by weight, preferably 3.0 to 7.0% by weight, of one or more isethionates of formula (1)

RCOOCH₂CH₂SO₃M (1)

in which
R is a linear or branched alkyl group having 6 to 30, preferably 8 to 22, carbon atoms or a linear or branched mono- or polyunsaturated alkenyl group having 6 to 30, preferably 8 to 22, carbon atoms and
M is a counterion, preferably an alkali metal, alkaline earth metal or ammonium ion,
B) 0.1 to 8.0% by weight, preferably 3.0 to 7.0% by weight, of one or more taurates of formula (2)

R¹CON(CH₃)CH₂CH₂SO₃X (2)

in which
R¹ is a linear or branched alkyl group or alkenyl group having 6 to 30, preferably 8 to 22, carbon atoms and
X is a counterion, preferably an alkali metal, alkaline earth metal or ammonium ion, and
C) 0.1 to 40.0% by weight, preferably 10.0 to 30.0% by weight, of one or more alkylbetaines of formula (3)

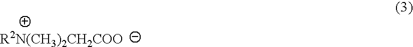

in which
R² is a linear or branched alkyl group or alkenyl group having 6 to 30, preferably 8 to 22, carbon atoms,
with the proviso that the concentration of the sum of all surfactants with formula 1, 2 and 3 is more than 20% by weight.
R, R¹ and R² in formulae (1), (2) and (3) are preferably the alkyl radicals of coconut or lauryl acid.

A particular advantage of the aqueous concentrates according to the invention is that they comprise the per se water-insoluble and solid surfactants taurate and isethionate in the form of a clear solution in a concentration which is several times above the solubility limit of these components in water. It is thus possible that these concentrates are stably incorporated into liquid end products such as, for example, hair shampoos, shower baths or liquid detergents, without a further heating or solubilization operation. It is to be expressly pointed out that not only does the concentrate of taurate, isethionate and betaine dissolve in aqueous surfactant mixtures to give a clear and homogeneous solution, but that the concentrate is as such homogeneous, transparent and of low viscosity. Furthermore, the stability of the concentrates according to the invention refers not only to a brief time window following preparation of the concentrate, but covers storage for three months at both high temperatures (40, 45 and 50° C.) and also low temperatures (−5, 0° C.).

A preferred embodiment of the invention is compositions comprising surfactants of formula (1), (2) and (3), where the amount by weight of these surfactants is together 20% to 49%, preferably 25% to 35%, particularly preferably 28% to 32%.

The surfactant concentrates according to the invention generally lie completely outside of the “G” phase.

A likewise preferred embodiment of the invention is compositions comprising surfactants of formula (1), (2) and (3), where the solids content of the compositions, consisting of the surfactants of formula (1), (2) and (3), and also fatty acids and salts, in particular NaCl, is 35 to 45%, preferably 40%.

A further preferred embodiment of the invention is compositions comprising surfactants of formula (1), (2) and (3), where the quantitative ratio of isethionate according to formula (1) to taurate according to formula (2) to betaine according to formula (3) is 0.5 to 1.5:0.5 to 1.5:4.

The transparency of the composition according to the invention is preferably max. 10 NTU (turbidity standard).

The color number of the composition according to the invention is max. 60 Hazen, preferably max. 30 Hazen, particularly preferably max. 20 Hazen.

A particularly preferred embodiment of the invention is aqueous concentrates comprising, based on the total concentrate,
A) 3.0 to 7.0% by weight of one or more isethionates of formula (1)

RCOOCH₂CH₂SO₃M (1)

in which
R is the hydrocarbon radical of coconut acid and
M is an alkali metal ion, in particular a sodium ion,
B) 3.0 to 7.0% by weight of one or more taurates of formula (2)

R¹CON(CH₃)CH₂CH₂SO₃X (2)

in which
R¹ is the hydrocarbon radical of coconut acid and
X is an alkali metal ion, in particular a sodium ion,
C) 15.0 to 25.0% by weight of one or more of alkylbetaines of formula (3)

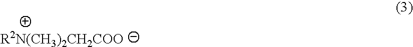

in which
R² is the hydrocarbon radical of coconut acid or of lauryl acid,
D) 5.0 to 9.0% by weight of sodium chloride and
E) 58 to 62% by weight of water and
F) 0.1 to 2% by weight, preferably 1% by weight, of fatty acid, preferably stearic or coconut fatty acid.

The compositions according to the invention are used for producing cosmetic, dermatological or pharmaceutical formulations, preferably for producing shower baths, shampoos or body cleansing compositions.

The invention therefore further provides the use of the compositions according to the invention (surfactant concentrates) in cosmetic, dermatological or pharmaceutical formulations.

The cosmetic, dermatological or pharmaceutical formulations comprising the surfactant concentrates according to the invention can comprise further surfactants, emulsifiers, cationic polymers, thickeners, film formers, antimicrobial active ingredients, astringents, antioxidants, UV photoprotective filters, pigments/micropigments, gelling agents, and also further additives customary in cosmetics, dermatology or pharmacy, such as, for example, superfatting agents, moisturizing agents, silicones, stabilizers, conditioners, glycerol, preservatives, pearlizing agents, dyes, enzyme inhibitors, solvents, hydrotropes, opacifiers, fatty alcohols, substances with keratolytic and keratoplastic effect, antidandruff agents, biogenic active ingredients (local anesthetics, antibiotics, antiphlogistics, antiallergics, corticosteroids, sebostatics), vitamins, Bisabolol®, allantoin, Phytantriol®, Panthenol®, AHA acids, plant extracts, for example aloe vera and proteins.

Anionic washing-active substances or surfactants that may be mentioned are preferably: C₁₀-C₂₀-alkyl- and alkylenecarboxylates, alkyl ether carboxylates, fatty alcohol sulfates, alkylamide sulfates and sulfonates, fatty acid alkylamide polyglycol ether sulfates, alkanesulfates, alkanesulfonates and hydroxyalkanesulfonates, olefinsulfonates, α-sulfo fatty acid esters, alkylbenzenesulfonates, alkylphenol glycol ether sulfonates, sulfosuccinates, sulfosuccinic acid half-esters and diesters, fatty alcohol ether phosphates, protein-fatty acid condensation products, alkyl monoglyceride sulfates and sulfonates, alkyl glyceride ether sulfonates, fatty acid sarcosinates, sulforicinoleates, amphoacetates or -glycinates, acylglutamates. These compounds and mixtures thereof are used in the form of their water-soluble or water-dispersible salts, for example the sodium, potassium, magnesium, ammonium, mono-, di- and triethanolammonium salts and also the analogous alkylammonium salts.

The amount of anionic surfactants in the formulations comprising the surfactant concentrates according to the invention is preferably 1 to 20% by weight, particularly preferably 5 to 18% by weight and especially preferably 10 to 15% by weight, based on the finished formulations.

Suitable cationic surfactants are, for example, quaternary ammonium salts, such as di(C₁₀-C₂₄-alkyl)dimethylammonium chloride or bromide, preferably di(C₁₂-C₁₈-alkyl)dimethylammonium chloride or bromide; C₁₀-C₂₄-alkyldimethylethylammonium chloride or bromide; C₁₀-C₂₄-alkyltrimethylammonium chloride or bromide, preferably cetyltrimethylammonium chloride or bromide and C₂₀-C₂₂-alkyltrimethylammonium chloride or bromide; C₁₀-C₂₄-alkyldimethylbenzylammonium chloride or bromide, preferably C₁₂-C₁₈-alkyldimethylbenzylammonium chloride; N—(C₁₀-C₁₈-alkyl)pyridinium chloride or bromide, preferably N—(C₁₂-C₁₆-alkyl)pyridinium chloride or bromide; N—(C₁₀-C₁₈-alkyl)isoquinolinium chloride, bromide or monoalkylsulfate; N—(C₁₂-C₁₈-alkylpolyoylaminoformylmethyl)pyridinium chloride; N—(C₁₂-C₁₈-alkyl)-N-methylmorpholinium chloride, bromide or monoalkylsulfate; N—(C₁₂-C₁₈-alkyl)-N-ethylmorpholinium chloride, bromide or monoalkylsulfate; C₁₆-C₁₈-alkylpentaoxyethylammonium chloride; diisobutylphenoxyethoxyethyldimethylbenzylammonium chloride; salts of N,N-diethylaminoethylstearylamide and oleylamide with hydrochloric acid, acetic acid, lactic acid, citric acid, phosphoric acid; N-acylaminoethyl-N,N-diethyl-N-methylammonium chloride, bromide or monoalkylsulfate and N-acylaminoethyl-N,N-diethyl-N-benzylammonium chloride, bromide or monoalkylsulfate, where acyl is preferably stearyl or oleyl.

The amount of cationic surfactants in the formulations comprising the surfactant concentrates according to the invention is preferably 0.1 to 10% by weight, particularly preferably 0.2 to 7% by weight, and especially preferably 0.5 to 5% by weight, based on the finished formulations.

Suitable nonionic surfactants which can be used as washing-active substances are preferably fatty alcohol ethoxylates (alkyl polyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (Pluronics®); fatty acid amide polyethylene glycols; N-alkyl-, N-alkoxypolyhydroxy fatty acid amide, in particular fatty acid N-methyl-glucamides, sucrose esters; polyglycol ethers, alkyl polyglycosides, phosphoric acid esters (mono-, di- and triphosphoric acid esters ethoxylated and nonethoxylated).

The amount of nonionic surfactants in the formulations comprising the surfactant concentrates according to the invention (e.g. in the case of rinse-off products) is preferably in the range from 1 to 20% by weight, particularly preferably 2 to 10% by weight, and especially preferably 3 to 7% by weight, based on the finished formulations.

Preferred amphoteric surfactants are: N—(C₁₂-C₁₈-alkyl)-β-aminopropionates and N—(C₁₂-C₁₈-alkyl)-β-iminodipropionates as alkali metal and mono-, di- and trialkylammonium salts; C₁₂-C₁₈-alkyldimethylsulfopropylbetaine; amphoteric surfactants based on imidazoline (trade name: Miranol®, Steinapon®), preferably the sodium salt of 1-(β-carboxymethyloxyethyl)-1-(carboxymethyl)-2-laurylimidazolinium; amine oxides, e.g. C₁₂-C₁₈-alkyldimethylamine oxide, fatty acid amidoalkyl-dimethylamine oxide.

The amount of amphoteric surfactants in the formulations comprising the surfactant concentrates according to the invention is preferably 0.5 to 20% by weight and particularly preferably 1 to 10% by weight, based on the finished formulations.

Furthermore, foam-boosting cosurfactants from the group alkylamidobetaines, aminopropionates, aminoglycinates, imidazoliniumbetaines and sulfobetaines, amine oxides and fatty acid alkanolamides or polyhydroxyamides can be used in the formulations comprising the surfactant concentrates according to the invention.

Preferred surfactants in the formulations comprising the surfactant concentrates according to the invention are alkyl ether sulfates, alkylsulfates, in particular laurylsulfate, alkylbetaines, alkylamidopropylbetaines, in particular cocoamidopropylbetaine, amphoacetates, acylglutamates, in particular sodium cocoylglutamate, alkyl ether sulfosuccinates, in particular disodium laureth sulfosuccinate, and coconut fatty acid diethanolamide.

The total amount of the surfactants used in the formulations comprising the surfactant concentrates according to the invention is preferably 1 to 20% by weight, particularly preferably 5 to 18% by weight, and especially preferably 10 to 15% by weight, based on the finished formulations.

Suitable cationic polymers are preferably the compounds known under the INCI name “Polyquaternium”, in particular Polyquaternium-31, Polyquaternium-16, Polyquaternium-24, Polyquaternium-7, Polyquaternium-22, Polyquaternium-39, Polyquaternium-28, Polyquaternium-2, Polyquaternium-10, Polyquaternium-11, Polyquaternium 37&mineral oil&PPG trideceth (Salcare® SC95), PVP-dimethylaminoethyl methacrylate copolymer, guar hydroxypropyltriammonium chlorides, and also calcium alginate and ammonium alginate.

Furthermore, use may preferably be made of cationic cellulose derivatives; cationic starch; copolymers of diallylammonium salts and acrylamides; quaternized vinylpyrrolidone/vinylimidazole polymers; condensation products of polyglycols and amines; quaternized collagen polypeptides; quaternized wheat polypeptides; polyethyleneimines; cationic silicone polymers, such as, for example, amidomethicones; copolymers of adipic acid and dimethylaminohydroxypropyldiethylene-triamine; polyaminopolyamide and cationic chitin derivatives, such as, for example, chitosan.

The amount of cationic polymers in the formulations can preferably be in the range from 0.1 to 10% by weight, particularly preferably in the range from 0.2 to 5% by weight, and especially preferably in the range from 0.5 to 2.5% by weight, based on the finished formulations.

Preferably suitable as antimicrobial active ingredients are cetyltrimethylammonium chloride, cetylpyridinium chloride, diisobutylethoxyethyidimethylbenzylammonium chloride, sodium N-laurylsarcosinate, sodium N-palmethylsarcosinate, lauroylsarcosine, N-myristoylglycine, potassium N-laurylsarcosine, trimethylammonium chloride, sodium aluminum chlorohydroxylactate, triethyl citrate, tricetylmethylammonium chloride, 2,4,4′-trichloro-2′-hydroxydiphenyl ether (triclosan), phenoxyethanol, 1,5-pentanediol, 1,6-hexanediol, 3,4,4′-trichlorocarbanilide (triclocarban), diaminoalkylamide, for example L-lysine hexadecylamide, citrate heavy metal salts, salicylates, piroctose, in particular zinc salts, pyrithiones and heavy metal salts thereof, in particular zinc pyrithione, zinc phenolsulfate, farnesol and combinations of these active substances.

The formulations comprise the antimicrobial agents preferably in amounts up to 50% by weight, particularly preferably in amounts of from 0.01 to 10% by weight, and especially preferably in amounts of from 0.1 to 10% by weight, based on the finished formulations.

Advantageous formulations comprise one or more antioxidants.

Water-soluble antioxidants can be used particularly advantageously for the purposes of the present invention.

The antioxidants can protect the skin and the hair against oxidative stress. Preferred antioxidants here are vitamin E and derivatives thereof, and vitamin A and derivatives thereof.

The amount of antioxidants (one or more compounds) in the formulations is preferably 0.001 to 30% by weight, particularly preferably 0.05 to 20% by weight, and in particular 1 to 10% by weight, based on the finished formulations.

Pigments/micropigments that can be used are preferably microfine titanium dioxide, mica-titanium oxide, iron oxides, mica-iron oxide, zinc oxide, silicon oxides, ultramarine blue, chromium oxides.

Further additives may be silicone compounds, preferably dimethylpolysiloxane, methylphenylpolysiloxanes, cyclic silicones, and also amino-, fatty-acid-, alcohol-, polyether-, epoxy-, fluorine- and/or alkyl-modified silicone compounds, for example alkylsilicones SilCare® Silicone 41M10, SilCare® Silicone 41M15, SilCare® Silicone 41M20, SilCare® Silicone 41M30 (Clariant), alkyltrimethicones SilCare® 31M30, SilCare® 31M40, SilCare® 31M50, SilCare® 31M60 (Clariant), phenyltrimethicones SilCare® 15M30, SilCare® 15M40, SilCare® 15M50, SilCare® 15M60 (Clariant), polyalkylarylsiloxanes and polyethersiloxane copolymers.

The formulations comprising the surfactant concentrates according to the invention can comprise the abovementioned silicone compounds preferably in the amounts of from 0.1 to 20% by weight, particularly preferably 0.2 to 15% by weight, especially preferably 0.5 to 10% by weight, based on the finished formulations.

Fungicidal active ingredients that can be used are preferably ketoconazole, oxiconazole, bifonazole, butoconazole, cloconazole, clotrimazole, econazole, enilconazole, fenticonazole, isoconazole, miconazole, sulconazole, tioconazole, fluconazole, itraconazole, terconazole, naftifine and terbinafine, Zn pyrithione and Octopirox® in the amounts from 0.05 to 5% by weight, preferably 0.1 to 3% by weight, and particularly preferably 0.2 to 2% by weight, based on the finished formulations.

The formulations comprising the surfactant concentrates according to the invention can advantageously be mixed with conventional ceramides, pseudoceramides, fatty acid N-alkylpolyhydroxyalkylamides, cholesterol, cholesterol fatty acid esters, fatty acids, triglycerides, cerebrosides, phospholipids and similar substances.

Preferred pearlizing compounds are fatty acid monoalkanolamides, fatty acid dialkanolamides, monoesters or diesters of alkylene glycol, in particular of ethylene glycol and/or propylene glycol or oligomers thereof with higher fatty acids, e.g. palmitic acid, stearic acid or behenic acid, or mixtures thereof, mono- or diesters of alkylene glycols with fatty acids, fatty acids and metal salts thereof, monoesters or polyesters of glycerol with carboxylic acids and ketosulfones of various types. In the formulations according to the invention, particularly preferred pearlizing components are ethylene glycol distearate and polyethylene glycol distearate with 3 glycol units.

Moisturizing substances that are available are preferably isopropyl palmitate, glycerol and/or sorbitol, which are preferably used in amounts of from 0.1 to 50% by weight, based on the finished formulations according to the invention.

Superfatting agents that can be used are preferably lanolin and lecithin, nonethoxylated and polyethoxylated or acylated lanolin and lecithin derivatives, polyol fatty acid esters, mono-, di- and triglycerides and/or fatty acid alkanolamides.

Suitable preservatives are preferably phenoxyethanol, parabens, pentanediol or sorbic acid. They are used preferably in amounts of from 0.001 to 5% by weight, particularly preferably from 0.01 to 3% by weight, and especially preferably from 0.1 to 2% by weight, based on the finished formulations according to the invention.

Dyes that can be used are the substances approved and suitable for cosmetic, dermatological and pharmaceutical purposes.

The total fraction of the auxiliaries and additives can be 1 to 50% by weight and preferably 5 to 40% by weight, based on the finished formulations. The formulations can be prepared by customary cold or hot processes.

The acids or alkalis used for adjusting the pH are preferably mineral acids, in particular HCl, inorganic bases, in particular NaOH or KOH, and organic acids, in particular citric acid.

The formulations comprising the surfactant concentrates according to the invention are adjusted preferably to a pH in the range 2 to 12 and particularly preferably to a pH in the range 3 to 8.

In a further particularly preferred embodiment of the invention, the formulations are formulations with a clear appearance.

The surfactant concentrates and compositions according to the invention are notable for very good foaming behavior and bring about a relatively large foam volume.

In the Ross Miles test and hand washing test, the foam properties of individual surfactants and also of surfactant concentrates according to the invention was tested. The overall surfactant concentration chosen in each case was 1.0%, 0.1% and 0.03%, the pH was adjusted to pH 7.0 and water with a German hardness of 15° was used. The amount of foam after 30 seconds and after 300 seconds was read off.

### Tables 1 to 4:

Foam height (mm) of the composition according to the invention compared to the individual components

### Hostapon® CT Paste (Sodium Methyl Cocoyl Taurate)

| | |
|---|---|
| Total surfactant | Time |

| concentration | 30 sec | 300 sec |
|---|---|---|
| 1% | 240 | 240 |
| 0.1% | 220 | 220 |
| 0.03% | 170 | 170 |

### Genagen® KB (Cocobetaine)

| | |
|---|---|
| Total surfactant | Time |

| concentration | 30 sec | 300 sec |
|---|---|---|
| 1% | 260 | 240 |
| 0.1% | 210 | 190 |
| 0.03% | 180 | 170 |

### Hostapon SCI 85 (Sodium Cocoyl Isethionate)

| | |
|---|---|
| Total surfactant | Time |

| concentration | 30 sec | 300 sec |
|---|---|---|
| 1% | 250 | 240 |
| 0.1% | 50 | 5 |
| 0.03% | 20 | 5 |

Surfactant concentrate according to the invention consisting of
20% cocobetaine
5% sodium methyl cocoyl taurate
5% sodium cocoyl isethionate

| | |
|---|---|
| Total surfactant | Time |

| concentration | 30 sec | 300 sec |
|---|---|---|
| 1% | 280 | 275 |
| 0.1% | 255 | 250 |
| 0.03% | 215 | 215 |

The surfactant concentrate according to the invention produces a larger amount of foam which is more stable, even after a longer waiting time, than when using the individual surfactants in the same concentration.

The surfactant concentrates and compositions according to the invention are colorless to very weakly colored and are notable for a low color number:

Surfactant concentrates according to the invention were evaluated according to the Pt—Co/Apha/Hazen color assessment. The three names are used in different areas of application but refer to the same method. The color assessment supplements the color assessment with iodine comparison solutions for weak colorations.

The principle of the color assessment consists in comparing the samples visually in standardized vessels with yellow standard solutions graded in concentration. For the Apha/Hazen/Pt-Co color number, according to a suggestion by A. Hazen in 1892, an acidic solution of potassium hexachloroplatinate(IV) and cobalt(II) chloride is used. A color number is assigned to the comparison solutions according to their platinum content in mg/l (range is 0-500). These solutions are also commercially available.

Several international and national standards describe the method in more detail, e.g.:

BS 5339:76 (1993) Measurement of Colour on Hazen Units/Platinum-Cobalt Scale
DIN 53409 Determination of the Hazen color number (APHA method)
DIN ISO 6271 Grading the color according to the platinum cobalt scale
   A: Surfactant concentrate according to the invention consisting of
20% Coco-Betaine
5% Sodium Methyl Cocoyl Taurate
5% Sodium Cocoyl Isethionate
Hazen color number: 5
   B: Surfactant concentrate according to the invention consisting of
10% Coco-Betaine
10% Sodium Methyl Cocoyl Taurate
10% Sodium Cocoyl Isethionate
Hazen color number: 18
   C: Reference mixture Miracare® UM-140 consisting of
Sodium Cocoyl Isethionate and Sodium Lauroamphoacetate and Sodium
Methyl Cocoyl Taurate and Propylene Glycol and Sodium Xylenesulfonate
Hazen color number: 380
   D: Reference mixture Miracare MS-2 consisting of:
PEG-80 Sorbitan Laurate and Sodium Trideceth Sulfate and PEG-150
Distearate and Cocamidopropyl Hydroxysultaine and Disodium
Lauroamphodiacetate and Sodium Laureth-13 Carboxylate
Hazen color number: 360
   E: Reference mixture Miracare MP-35 consisting of:
Sodium Laureth Sulfate and Cocamide DEA and Cocamidopropyl Betaine and Citric Acid
Hazen color number: 290

The examples and applications below are intended to illustrate the invention in more detail without, however, limiting it thereto (all of the percentage data are % by weight).

The composition according to the invention used was surfactant concentrate A consisting of:
20% Coco-Betaine
5% Sodium Methyl Cocoyl Taurate
5% Sodium Cocoyl Isethionate

### EXAMPLES

### Example 1

### Care Shampoo

| | | | |
|---|---|---|---|
| A | Genapol ® LRO liq. | (Clariant) | 33.30% |
| | Laureth sulfate, Na salt | | |
| | Fragrance | | 0.40% |
| B | Water | | 20.00% |
| | Ucare Polymers ® JR 400 | | 0.30% |
| | Polyquaternium-10 | | |
| C | Water | | ad 100% |
| | Allantoin | (Clariant) | 0.30% |
| | Allantoin | | |
| D | Surfactant concentrate A | | 20.00% |
| | Cocobetaine, sodium cocoyl isethionate, | | |
| | sodium methyl cocoyl taurate | | |
| | Dye solution | | q.s. |
| | Preservative | | q.s. |
| | D-Panthenol | | 0.50% |
| E | Citric acid (25%) | | q.s. |

Preparation procedure
I Mixing of component A
II Dissolution of B at 80° C.
III Dissolution of C at 80° C.
IV Successive stirring in of II and III into I
V Addition of D to IV
VI Adjustment of the pH with E to 5.5

### Example 2

### Clear Antidandruff Shampoo

| | | | |
|---|---|---|---|
| A | Genapol ® LRO liq. | (Clariant) | 38.90% |
| | Laureth sulfate, Na salt | | |
| | Fragrance | | 0.40% |
| | Octopirox ® | (Clariant) | 0.50% |
| | Piroctone olamine | | |
| B | Water | | ad 100% |
| | Surfactant concentrate A | | 16.00% |
| | Cocobetaine, sodium cocoyl isethionate, | | |
| | sodium methyl cocoyl taurate | | |
| | Dye solution | | q.s. |
| C | Citric acid (25%) | | q.s. |

Preparation procedure:
I Mixing of component A
Successive addition of components B to I
III Adjustment of the pH with C to 5.5

### Example 3

### Care Shampoo

| | | |
|---|---|---|
| A | Ucare Polymer ® JR 400 | 0.40% |
| | Polyquaternium-10 | |
| | Water | ad 100% |
| B | Surfactant concentrate A | 8.00% |
| | Cocobetaine, sodium cocoyl isethionate, sodium | |
| | methyl cocoyl taurate | |
| | Genapol ® LRO liq. | 35.00% |
| | Laureth sulfate, Na salt | |
| | Belsil DMC 6032 | 0.50% |
| | Dimethicone PEG-6 acetate | |
| | Genagen ® KB | 3.50% |
| | Cocobetaine | |
| C | Glycerol | 2.00% |
| | Sorbitol | 2.00% |
| | Mackpro SLP | 0.50% |
| | Quaternium-79 hydrolyzed soy protein | |
| D | Citric acid 25% strength | q.s. |
| E | Preservative | q.s. |
| | Dye solution | q.s. |
| | Fragrance | 0.30% |
| F | Sodium chloride | 1.50% |

Preparation procedure:
I Dissolution of A at 80° C.
II Mixing of the components of B
III Successive addition of C to I

IV Stirring in of III into II; stirring until clear
V Adjustment of the pH with D to pH 5.5
VI Addition of E
VII Adjustment of the viscosity with F

### Example 4

### Face Cleansing Composition

| | | | |
|---|---|---|---|
| A | Genapol ® LRO liq. | (Clariant) | 11.10% |
| | Laureth sulfate, Na salt | | |
| | Fragrance | | 0.40% |
| B | Water | | ad 100% |
| | Surfactant concentrate A | | 23.30% |
| | Cocobetaine, sodium cocoyl isethionate, | | |
| | sodium methyl cocoyl taurate | | |
| | Dye solution | | q.s. |
| | Preservative | | q.s. |
| C | Citric acid | | q.s. |

Preparation procedure:
I Mixing of the components of A
II Addition of B to I
III Adjustment of the pH with C

### Example 5

### Antiacne Face Cleansing Composition

| | | | |
|---|---|---|---|
| A | Genapol ® LRO liq. | (Clariant) | 16.00% |
| | Laureth sulfate, Na salt | | |
| | Fragrance | | 0.40% |
| | Octopirox ® | (Clariant) | 0.50% |
| | Piroctone olamine | | |
| B | Allantoin | (Clariant) | 0.30% |
| | Allantoin | | |
| C | Water | | ad 100% |
| D | Surfactant concentrate A | | 16.00% |
| | Cocobetaine, sodium cocoyl isethionate, | | |
| | sodium methyl cocoyl taurate | | |
| | Dye solution | | q.s. |
| E | Citric acid (25%) | | q.s. |

Preparation procedure
I Mixing of the components of A
II Dissolution of B in heated C; cooling with stirring
III Addition of II to I
IV Addition of D to III
V Adjustment to pH 5.5 with E.

1. An aqueous concentrate comprising, based on the total composition,
A) 0.1 to 8.0% by weight of at least one isethionate of formula (1)
RCOOCH₂CH₂SO₃M (1)
wherein
R is a linear or branched alkyl group having 6 to 30 carbon atoms or a linear or branched mono- or polyunsaturated alkenyl group having 6 to 30 carbon atoms and
M is a counterion,
B) 0.1 to 8.0% by weight of at least one taurate of formula (2)
R¹CON(CH₃)CH₂CH₂SO₃X (2)
wherein which
R¹ is a linear or branched alkyl group having 6 to 30 carbon atoms or a linear or branched mono- or polyunsaturated alkenyl group having 6 to 30 carbon atoms and
X is a counterion,
C) 0.1 to 40.0% by weight of at least one alkylbetaine of formula (3) 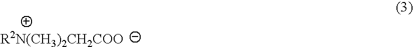
wherein
R² is a linear or branched alkyl group having 6 to 30 carbon atoms or a linear or branched mono- or polyunsaturated alkenyl group having 6 to 30 carbon atoms, with the proviso that the concentration of the sum of all surfactants of the formula 1, 2 and 3 is more than 20% by weight.

2. The aqueous concentrate as claimed in claim 1, comprising compounds of formulae (1), (2) and (3), wherein R, R¹ or R² is the alkyl radical of coconut or lauryl acid.

3. The aqueous concentrate as claimed in claim 1, wherein the total amount by weight of all surfactants of formulae (1), (2) and (3) is 20 to 49%.

4. The aqueous concentrate as claimed in claim 1, wherein the total amount by weight of all surfactants of formulae (1), (2) and (3) is 25 to 35%.

5. The aqueous concentrate as claimed in claim 1, wherein the total amount by weight of all surfactants of formulae (1), (2) and (3) is 28 to 32%.

6. The aqueous concentrate as claimed in claim 1, wherein the total solids content, consisting of the surfactants of formulae (1), (2) and (3), and acids and salts, is 35 to 45% by weight.

7. The aqueous concentrate as claimed in claim 1, where the quantitative ratio of the compounds of formulae (1), (2) and (3) is 0.5 to 1.5:0.5 to 1.5:4.

8. The aqueous concentrate as claimed in claim 1, which has a color number of at most 60 Hazen.

9. The aqueous concentrate as claimed in claim 1, which has a transparency of at most 10 NTU.

10. An aqueous concentrate comprising, based on the total composition,
A) 3.0 to 7.0% by weight of at least one isethionate of formula (1)
RCOOCH₂CH₂SO₃M (1)
wherein
R is the hydrocarbon radical of coconut acid and
M is an alkali metal ion,
B) 3.0 to 7.0% by weight of at least one taurate of formula (2)
R¹CON(CH₃)CH₂CH₂SO₃X (2)
wherein
R¹ is the hydrocarbon radical of coconut acid and
X is an alkali metal ion,
C) 15.0 to 25.0% by weight of at least one of alkylbetaine of formula (3) 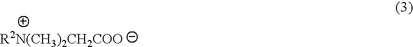
wherein
R² is the hydrocarbon radical of coconut acid,
D) 5.0 to 9.0% by weight of sodium chloride,
E) 58 to 62% by weight of water and
F) 0.1 to 2% by weight, of fatty acid,

11. A cosmetic, dermatological or pharmaceutical formulation comprising an aqueous concentrate as claimed in claim 1.
